(19) United States
(12) Patent Application Publication
(10) Pub. No.: US 2026/0127735 A1
(43) Pub. Date: May 7, 2026

(54) DEEP LEARNING ESTIMATION OF VASCULAR FLOW AND PROPERTIES

(71) Applicant: The Johns Hopkins University, Baltimore, MD (US)

(72) Inventors: Bharath AMBALE VENKATESH, Baltimore, MD (US); Joao LIMA, Baltimore, MD (US)

(73) Assignee: The Johns Hopkins University, Baltimore, MD (US)

(21) Appl. No.: 18/704,336

(22) PCT Filed: Nov. 3, 2022
(86) PCT No.: PCT/US2022/048854

§ 371 Date: Apr. 24, 2024

Related U.S. Application Data
(60) Provisional application No. 63/277,408, filed on Nov. 9, 2021.

(51) Int. Cl. G06T 7/00; G06T 7/10; G16H 30/20; G16H 30/40

(52) U.S. Cl. CPC G06T 7/0012; G06T 7/10; G16H 30/20; G16H 30/40; G06T 2207/10088; G06T 2207/20081; G06T 2207/30104

(57) ABSTRACT

A method for characterizing a blood vessel includes receiving anatomical information of the blood vessel and receiving four-dimensional flow data of multiple blood vessels from multiple subjects. The method further includes, using the four-dimensional flow data to train a flow model to determine blood vessel properties, and using the anatomical information of the blood vessel as an input to the trained flow model to estimate one or more properties of the blood vessel.

### CROSS-REFERENCE OF RELATED APPLICATION

This application claims priority to U.S. Provisional Application No. 63/277,408, filed Nov. 9, 2021, which is incorporated herein by reference in its entirety.

### BACKGROUND

### 1. Technical Field

Embodiments of this disclosure relate to systems and methods for measurement of vascular flow, and more particularly quantitative properties of blood vessels.

### 2. Discussion of Related Art

Atlas-based morphometry, a concept that refers to the computational analysis of form, is a method of quantifying the major determinants of remodeling at the regional and global levels and may aid in the precise quantification of subclinical disease.¹⁻³ Although these methods are well established in neuroimaging to identify brain anatomical and functional regions, application to the dynamic cardiovascular system has been limited.⁴ Shape captures an ensemble of aspects associated with the coupling of alterations in cardiac structure with function that can be used to phenotype disease.⁵

Elevated central arterial stiffness, a hallmark of aging, is associated with adverse clinical outcomes, including coronary heart disease, stroke, and cardiovascular disease (CVD) mortality.⁶⁻⁸ Human studies have shown that greater arterial stiffness is closely related with both systolic and diastolic dysfunction,⁹⁻¹¹ and individuals with heart failure (HF) have elevated arterial stiffness.¹²⁻¹⁴ Comprehensive prospective studies relating arterial stiffness to HF risk in large populations are sparse. The pathogenesis of aortic stiffness-related CVD may be due but also potentiate abnormal aortic shape, particularly in patients with HFpEF.¹⁵ Aortic shape has been analyzed before in the setting of specific aortopathies,¹⁶⁻¹⁸ and is seen to be a key influencer of blood flow patterns through the aorta.¹⁹ Abnormal blood flow patterns and increased wall shear stress are associated with LV remodeling. One recent study has shown that the magnitude of ascending aorta backward flow increases with age and its onset occurs earlier in the cardiac cycle, during LV ejection, with aging.²⁰ Such phenomena are strongly associated with geometric changes of the aortic arch such as dilation and elongation that lead to changes in local gradients in pressure and wall shear stress.^{19,21}

Currently, there are two common methods to perform a comprehensive assessment of vascular flow and stiffness. One technique involves imaging the vascular anatomy followed by the use of computational fluid dynamics (CFD) to assess vascular tissue (wall) properties through the use of time-consuming simulations, which require additional specialized software and may also require specially trained engineering specialists. Another technique involves a comprehensive imaging strategy (4D flow) which is followed by a time-consuming image analysis procedure.

4D flow MRI has greatly expanded the capability to study the pathophysiologic pathways associated with arterial and venous flow and remodeling. The advantage of 4D flow MRI is that full information of flow mechanics is obtained, including full spatial-temporal coverage of the aorta and the estimation of intra-aortic pressure gradients, and flow:²²⁻²⁴ Full 3D wall shear stress estimates can be generated as well. Abnormal blood flow patterns and increased wall shear stress of the aorta (specifically) are associated with LV remodeling and may form important aspects of intervention in heart failure.

However, 4D flow MRI has a number of disadvantages. These include long acquisition times (e.g., 5-20 min), which can cause problems with breathing patterns and movement. There is no online post-processing (e.g., on the scanner), and instead needs special software. The post-processing also requires large memory as there needs to be ability to deal with large datasets. Furthermore, 4D flow MRI has lower temporal resolution as compared to 2D phase-contrast MRI.

The complexity of 4D flow post-processing requirements have resulted in limited use, particularly in large population studies, in spite of recent advances to both improve post-processing time as well as resolution.²⁵⁻²⁷ For these reasons, it is difficult to assess the quality of scan when the patient is on the table.

### SUMMARY

An embodiment of the present invention is a method for characterizing a blood vessel. The method includes receiving anatomical information of the blood vessel and receiving four-dimensional flow data of multiple blood vessels from multiple subjects. The method further includes using the four-dimensional flow data to train a flow model to determine blood vessel properties, and using the anatomical information of the blood vessel as an input to the trained flow model to estimate one or more properties of the blood vessel.

Another embodiment of the present invention is a system for characterizing a blood vessel, including an imaging system and a data processor. The data processor is configured to communicate with the imaging system to receive anatomical information of the blood vessel and receive four-dimensional flow data of multiple blood vessels from multiple subjects. The data processor is further configured to use the four-dimensional flow data to train a flow model to determine blood vessel properties, and use the anatomical information of the blood vessel as an input to the trained flow model to estimate one or more properties of the blood vessel.

Another embodiment of the present invention is a non-transitory machine-readable medium storing a program for characterizing a blood vessel, which when executed by a data processor, configures a data processor. The data processor is configured by the executed program to communicate with the imaging system to receive anatomical information of the blood vessel and receive four-dimensional flow data of multiple blood vessels from multiple subjects. The data processor is further configured by the executed program to use the four-dimensional flow data to train a flow model to determine blood vessel properties, and use the anatomical information of the blood vessel as an input to the trained flow model to estimate one or more properties of the blood vessel.

### BRIEF DESCRIPTION OF THE DRAWINGS

Further objectives and advantages will become apparent from a consideration of the description, drawings, and examples.

FIG. 1A conceptually illustrates a system of some embodiments for using deep-learning methods to perform flow-related computations.

FIG. 1B conceptually illustrates an example where a data processor of the system in FIG. 1A is configured with a flow model.

FIG. 1C conceptually illustrates an example where the imaging system includes an image processor and one or more imaging devices.

FIG. 2 shows a visual comparison of point-wise peak wall shear stress estimates from a conventional 4D flow MRI image and a deep-learning image generated by a trained flow model in some embodiments.

FIG. 3 illustrates an example of a multi-layer machine-trained network that can be trained and used as a model for deep learning in some embodiments.

### DETAILED DESCRIPTION

Some embodiments of the invention are discussed in detail below. In describing embodiments, specific terminology is employed for the sake of clarity. However, the invention is not intended to be limited to the specific terminology so selected. A person skilled in the relevant art will recognize that other equivalent components can be employed, and other methods developed, without departing from the broad concepts of the current invention.

All references cited anywhere in this specification, including the Background and Detailed Description sections, are incorporated by reference as if each had been individually incorporated.

As used herein, the term “deep learning” (DL) refers to artificial intelligence and/or machine learning techniques including, but not limited to, convolutional neural networks. Deep-learning (DL) based simulations to quantify fractional flow-reserve have been used in the setting of coronary artery disease with high accuracy.²⁸ However, such DL techniques have not previously been applied to quantification of central arterial stiffness.

As used herein, the term “4D flow” refers to volumetric data acquired over a period of time and processed to determine three-dimensional velocity encoding. In other words, 4D flow data characterizes the temporal evolution of complex blood flow patterns within an acquired 3D volume.

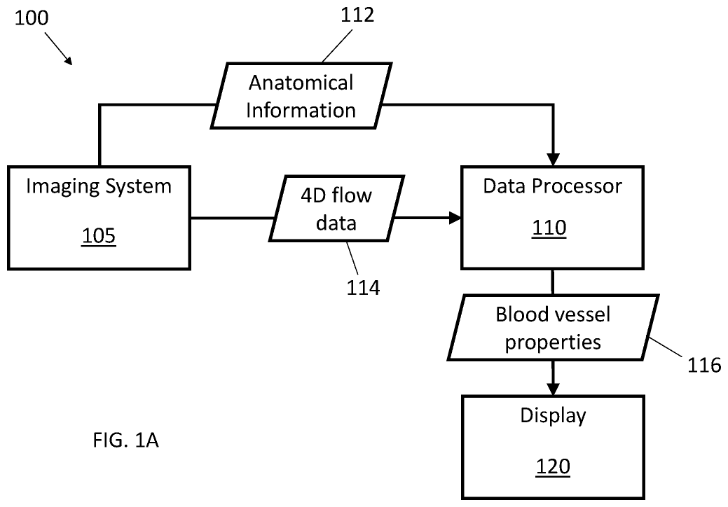

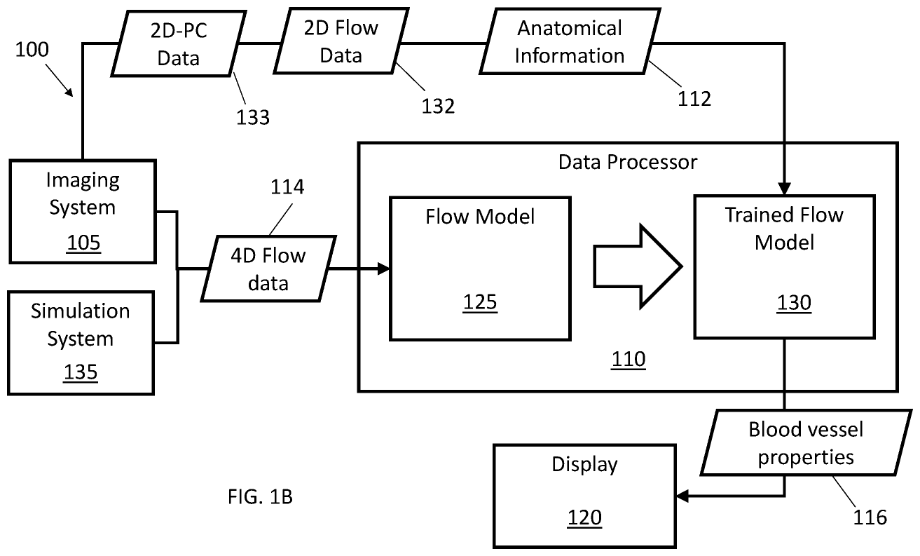

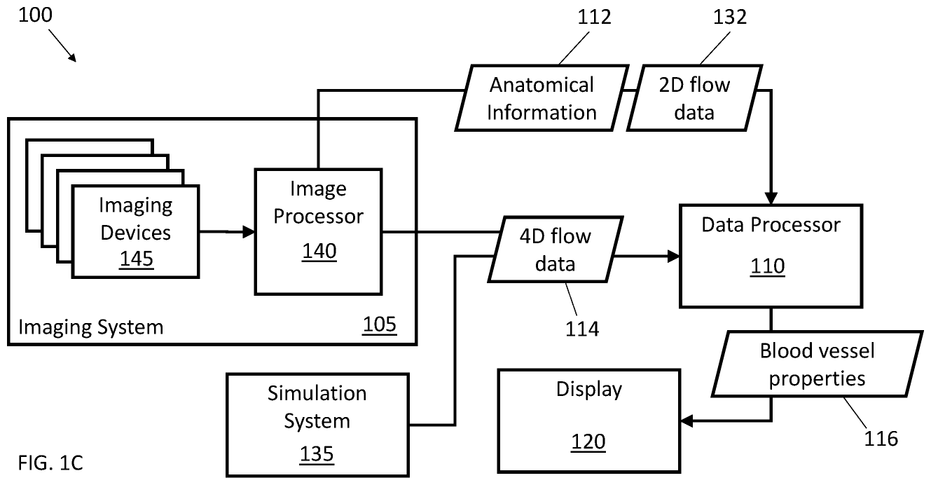

FIG. 1A conceptually illustrates a system 100 of some embodiments for using deep-learning methods to perform flow-related computations. In some embodiments, the system 100 applies DL for characterizing blood vessels, including but not limited to assessment of full aortic four-dimensional (4D) flow and wall shear stress assessment, using three-dimensional (3D) aortic shapes and ascending aortic waveforms as input flow. In some embodiments, these techniques may reduce the acquisition time to less than a minute and the post-processing time to a few seconds.

In the example of FIG. 1A, the system 100 includes an imaging system 105 and a data processor 110 that is configured to communicate with the imaging system 105 to receive anatomical information 112 of a blood vessel (not shown) from a current subject (e.g., a patient), and four-dimensional (4D) flow data 114 of multiple blood vessels from multiple previous subjects. The data processor 110 is further configured to use the anatomical information 112 and 4D flow data 114 to estimate properties 116 of the blood vessel. The system 100 may also include a display 120 in some embodiments, to provide the blood vessel properties 116 to a user (not shown) of the system 100, such as a physician.

The blood vessel may be any blood vessel (e.g., an artery or a vein) in any part of the body. In some embodiments, the blood vessel may be any of the aorta, the coronary artery, the carotid artery, the superior vena cava, and the inferior vena cava. The vessels of interest may include (but are not limited to) large and small arteries and veins, cardiac vessels, and brain vessels. However, the blood vessel is not limited to these vessels.

In some embodiments, the anatomical information 112 includes, but is not limited to, shape information of the blood vessel. The anatomical information 112 of the blood vessel may be generated in some embodiments from a segmentation process applied to one or more images of the blood vessel that are acquired using the imaging system 105.

In some embodiments, the blood vessel properties 116 include but are not limited to at least one of the wall shear stress of the blood vessel expressed in units of force per unit area, the wall stiffness of the blood vessel expressed in units of pulse wave velocity, the vorticity of the blood vessel expressed in units of inverse time, the velocity of blood within the blood vessel expressed in units of distance over time, and the flow of blood within the blood vessel expressed in units of volume over time.

In some embodiments, flow and vascular wall properties are computed (e.g., by the data processor 110) directly from the imaging anatomy (e.g., the anatomical information 112) using deep learning (DL). The vascular anatomy of interest and output comprehensive flow and vascular wall property maps are used as inputs to the network. Additional inputs such as inlet and outlet boundary conditions may also be added to make the models more robust. Additional outputs that directly produce quantitative measurements such as vorticity, shear stress, velocity, and flow may also be added to make the model more comprehensive and directly usable in the clinical scenario.

FIG. 1B conceptually illustrates an example where the data processor 110 of the system 100 in FIG. 1A is configured with a flow model 125. The 4D flow data 114 is used by a training process (not shown) in some embodiments to train the flow model 125 to determine the blood vessel properties 116. This training process transforms the flow model 125 to a trained flow model 130.

In some embodiments, the flow model 125 includes a neural network, and the 4D flow data 114 is used to train the neural network to obtain quantitative vessel properties and measurements.

The anatomical information 112 of the blood vessel is used as an input to the trained flow model 130, and the blood vessel properties 116 of the blood vessel are an output of the trained flow model 130. In some embodiments, the data processor 110 is further configured to receive from the imaging system 105 one or more measurements of two-dimensional (2D) flow data 132 of blood that enters or exits the blood vessel. The 2D flow data 132 may be used as a further input to the trained flow model 130. In some embodiments, only the shape of the specific vessels is used as input to the trained flow model 130.

The anatomical information 112, including but not limited to the shape of a vessel, may be obtained from images acquired with any imaging modality, including but not limited to computed tomography (CT), ultrasound, and magnetic resonance imaging (MRI). For example, the anatomical information 112 may be axial slices acquired using steady-state free precession (SSFP) MRI (e.g., having a ˜10 sec acquisition time). In addition, in some embodiments, one or two slices of two-dimensional phase contrast (2D-PC) data 133 may also be obtained at the ascending or descending arms of the aorta and used as input to the trained flow model 130.

An initial pre-processing step of segmentation of the vascular anatomy may also be used in some embodiments to further aid in building a comprehensive end-to-end solution. As an example, a DL-based segmentation algorithm may be used in some embodiments to segment the aorta or any other vessel of interest.

In some embodiments, at least a portion of the 4D flow data 114 may be acquired using the imaging system 105.

In some embodiments, the flow model 125 is trained based both on simulations performed offline using a computational fluid dynamics (CFD) model, as well as actual flow data obtained from the trained flow model 130. As an example, in some embodiments, at least a portion of the 4D flow data 114 may be simulated flow data acquired from a simulation system 135. The simulated data may be generated by the simulation system 135 at least in part based on flow data acquired from previous subjects.

FIG. 1C conceptually illustrates an example where the imaging system 105 includes an image processor 140 and one or more imaging devices 145. The imaging devices 145 may include, but are not limited to, a doppler ultrasound scanner, a computed tomography scanner, and a magnetic resonance scanner. In some embodiments, the image processor 140 receives imaging data (not shown) from the imaging devices 145 and processes that imaging data to generate at least one of the anatomical information 112, the 4D flow data 114, and the 2D flow data 132.

In some embodiments, one or both of the anatomical information 112 and the 4D flow data 114 may include magnetic resonance imaging data (not shown) acquired from an MRI scanner of the imaging devices 145. The magnetic resonance imaging data may be acquired with any pulse sequence, including but not limited to steady-state free procession (SSFP), dark blood sequences, bright blood sequences, gradient-recalled echo (GRE) sequences, and spin echo (SE) sequences such as fast spin echo (FSE) sequences.

The system 100 provides several advantages over the above-mentioned CFD or 4D flow techniques in several ways. For example, the system 100 may use vascular anatomy obtained from any imaging modality, and from any of the many sequences available, as opposed to merely phase-contrast MRI as in the case of 4D flow. This flexibility reduces the dependence on MRI, which is significantly more expensive than other imaging modalities. Moreover, the time of acquisition with the system 100 (on the order of seconds) imposes a lower burden on the patients, as opposed to 5-15 minutes as in the case of 4D flow MRI.

In addition, the system 100 does not require the use of complex CFD software to simulate and compute vascular properties (as in the case of CFD). This reduces the burden of the necessary computational power needed. Furthermore, it is not required to handle large amounts of data as would be the case in 4D flow imaging or the CFD method. Relative to 4D flow imaging with MRI or CFD simulations, the system 100 may utilize less hardware storage space. Finally, considering the amount of time and computation needs, results can be obtained inline, and available in a matter of seconds after acquisition.

As discussed above, some embodiments use deep learning (DL) based methods to overcome the need for complex 4D flow MRI processes, in the analysis of aortic wall shear stress and 4D flow patterns based on the shape of the aorta and an input function. The input function may be, for example, the flow at the ascending aorta. Deep learning can be applied to aortic shape databases to fully phenotype multiple participants with aortic wall shear stress and flow estimates. These results can be used to test the hypothesis that aortic wall shear stress and flow patterns directly relate to LV remodeling and are associated with heart failure and cardiovascular disease development, as has been posited based on data from small observational studies.^{29,30}

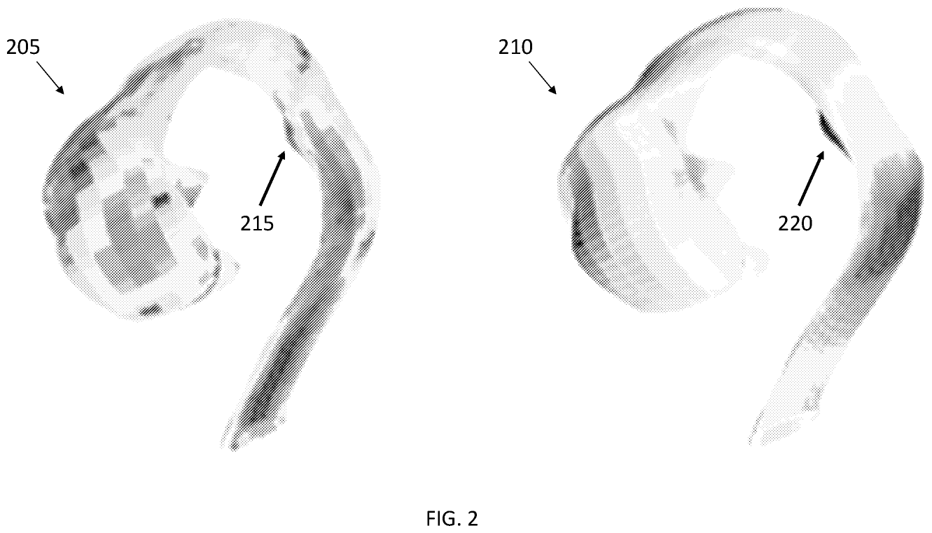

FIG. 2 shows a visual comparison of point-wise peak wall shear stress estimates from (a) a conventional 4D flow MRI image 205 (left), and (b) a deep-learning image 210 (right) generated by the trained flow model 130 in some embodiments. The results illustrate preliminary data of one patient taken from a group of 25 patients with dilated ascending aortas, with either a bicuspid or tricuspid aortic valve. An aneurysm is identifiable in a region 215 on the 4D flow MRI image 205, and is also identifiable in a region 220 on the deep-learning image 210.

In the example of FIG. 2, a PointNet architecture was used with an additional input with ascending aortic flow. The PointNet architecture has several unique advantages, namely that (1) there is no need to convert the aortic shape features present in the form of a point cloud to a 3D volume, (2) convolutions are performed with the actual shape as the basis, and (3) the input point-cloud for this architecture does not have to be parametric-therefore unordered and unequally spaced points can be used as input.^{31,32} These advantages are important considering (a) the aortic meshes created using diffeomorphic mapping are unordered and nonparametric, (b) wall shear stress is typically calculated at the points on the aortic wall, and (c) flow patterns are assessed within the shape of the aortic wall.

For the results shown in FIG. 2, a leave-one-out train/validation scheme was used where each subject was used as the test data with the remaining as the training data (considering the small sample size used here). A parametric mesh with 4096 points (128 longitudinal and 32 cross-sectional across the length of the aorta) was constructed and all 4D flow analysis was performed as previously described using ARTFUN software.²³ The preliminary results show that the DL-estimated wall shear stress and the 4D flow-based wall shear stress calculated at systole (time of peak wall shear stress) was moderate (r=0.77, p<0.01). Considering the small sample size and lack of data from normal volunteers in this example, results will be adequately accurate when a larger dataset is used for training the network.

The deep learning network of some embodiments is a multi-layer machine-trained network (e.g., a feed-forward neural network). Neural networks, also referred to as machine-trained networks, will be herein described. One class of machine-trained networks are deep neural networks with multiple layers of nodes. Different types of such networks include feed-forward networks, convolutional networks, recurrent networks, regulatory feedback networks, radial basis function networks, long-short term memory (LSTM) networks, and Neural Turing Machines (NTM). Multi-layer networks are trained to execute a specific purpose, including face recognition or other image analysis, voice recognition or other audio analysis, large-scale data analysis (e.g., for climate data), etc. In some embodiments, such a multi-layer network is designed to execute on a mobile device (e.g., a smartphone or tablet), an IOT device, a web browser window; etc.

A typical neural network operates in layers, each layer having multiple nodes. In convolutional neural networks (a type of feed-forward network), a majority of the layers include computation nodes with a (typically) nonlinear activation function, applied to the dot product of the input values (either the initial inputs based on the input data for the first layer, or outputs of the previous layer for subsequent layers) and predetermined (i.e., trained) weight values, along with bias (addition) and scale (multiplication) terms, which may also be predetermined based on training. Other types of neural network computation nodes and/or layers do not use dot products, such as pooling layers that are used to reduce the dimensions of the data for computational efficiency and speed.

For convolutional neural networks that are often used to process electronic image and/or video data, the input activation values for each layer (or at least each convolutional layer) are conceptually represented as a three-dimensional array. This three-dimensional array is structured as numerous two-dimensional grids. For instance, the initial input for an image is a set of three two-dimensional pixel grids (e.g., a 1280×720 RGB image will have three 1280×720 input grids, one for each of the red, green, and blue channels). The number of input grids for each subsequent layer after the input layer is determined by the number of subsets of weights, called filters, used in the previous layer (assuming standard convolutional layers). The size of the grids for the subsequent layer depends on the number of computation nodes in the previous layer, which is based on the size of the filters, and how those filters are convolved over the previous layer input activations. For a typical convolutional layer, each filter is a small kernel of weights (often 3×3 or 5×5) with a depth equal to the number of grids of the layer's input activations. The dot product for each computation node of the layer multiplies the weights of a filter by a subset of the coordinates of the input activation values. For example, the input activations for a 3×3×Z filter are the activation values located at the same 3×3 square of all Z input activation grids for a layer.

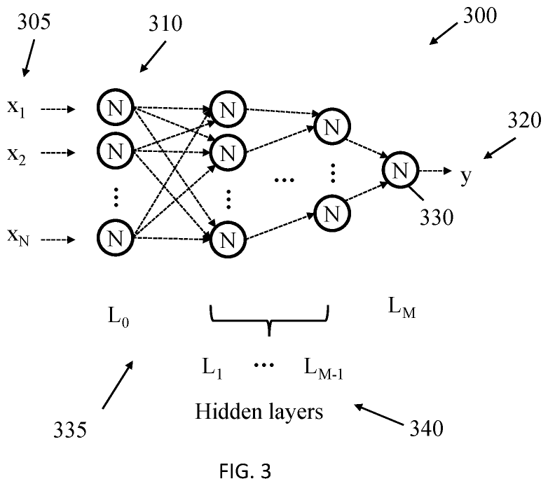

FIG. 3 illustrates an example of a multi-layer machine-trained network that can be trained and used as a model for deep learning in some embodiments. Specifically, the example of FIG. 3 illustrates a feed-forward neural network 300 that receives an input vector 305 (denoted x1, x2, . . . xN) at multiple input nodes 310 and computes an output 320 (denoted by y) at an output node 330. In some embodiments, the data processor 110 may be configured to execute the neural network 300 such that before training, the neural network serves as the flow model 125, and after training, the neural network serves as the trained flow model 130.

The neural network 300 has multiple layers L0, L1, L2 . . . . LM 335 of processing nodes (also called neurons, each denoted by N). In all but the first layer (input, L0) and last layer (output, LM), each node receives two or more outputs of nodes from earlier processing node layers and provides its output to one or more nodes in subsequent layers. These layers are also referred to as the hidden layers 340. Though only a few nodes are shown in FIG. 3 per layer, a typical neural network may include a large number of nodes per layer (e.g., several hundred or several thousand nodes) and significantly more layers than shown (e.g., several dozen layers). The output node 330 in the last layer computes the output 320 of the neural network 300.

In this example, the neural network 300 only has one output node 330 that provides a single output 320. Other neural networks of other embodiments have multiple output nodes in the output layer LM that provide more than one output value. In different embodiments, the output 320 of the network is a scalar in a range of values (e.g., 0 to 1), a vector representing a point in an N-dimensional space (e.g., a 128-dimensional vector), or a value representing one of a predefined set of categories (e.g., for a network that classifies each input into one of eight possible outputs, the output could be a three-bit value).

Portions of the illustrated neural network 300 are fully-connected in which each node in a particular layer receives as inputs all of the outputs from the previous layer. For example, all the outputs of layer L0 are shown to be an input to every node in layer L1. The neural networks of some embodiments are convolutional feed-forward neural networks, where the intermediate layers (referred to as “hidden” layers) may include other types of layers than fully-connected layers, including convolutional layers, pooling layers, and normalization layers.

The convolutional layers of some embodiments use a small kernel (e.g., 3×3×3) to process each tile of pixels in an image with the same set of parameters. The kernels (also referred to as filters) are three-dimensional, and multiple kernels are used to process each group of input values in in a layer (resulting in a three-dimensional output). Pooling layers combine the outputs of clusters of nodes from one layer into a single node at the next layer, as part of the process of reducing an image (which may have a large number of pixels) or other input item down to a single output (e.g., a vector output). In some embodiments, pooling layers can use max pooling (in which the maximum value among the clusters of node outputs is selected) or average pooling (in which the clusters of node outputs are averaged).

Each node computes a dot product of a vector of weight coefficients and a vector of output values of prior nodes (or the inputs, if the node is in the input layer), plus an offset. In other words, a hidden or output node computes a weighted sum of its inputs (which are outputs of the previous layer of nodes) plus an offset (also referred to as a bias). Each node then computes an output value using a function, with the weighted sum as the input to that function. This function is commonly referred to as the activation function, and the outputs of the node (which are then used as inputs to the next layer of nodes) are referred to as activations.

Consider a neural network with one or more hidden layers 340 (i.e., layers that are not the input layer or the output layer). The index variable l can be any of the hidden layers of the network (i.e., l∈{1, . . . , M−1}, with l=0 representing the input layer and l=M representing the output layer).

The output yₗ₊₁ of node in hidden layer l+1 can be expressed as:

$${y}_{l + 1} = f ( ( {w}_{l + 1} \cdot {y}_{l} ) * c + {b}_{l + 1} ) \qquad ( 1 )$$

This equation describes a function, whose input is the dot product of a vector of weight values wₗ₊₁ and a vector of outputs yr from layer l, which is then multiplied by a constant value c, and offset by a bias value bₗ₊₁. The constant value c is a value to which all the weight values are normalized. In some embodiments, the constant value c is 1. The symbol * is an element-wise product, while the symbol · is the dot product. The weight coefficients and bias are parameters that are adjusted during the network's training in order to configure the network to solve a particular problem (e.g., object or face recognition in images, voice analysis in audio, depth analysis in images, etc.).

In equation (1), the function ƒ is the activation function for the node. Examples of such activation functions include a sigmoid function (ƒ(x)=1/(1+e^{-x})), a tanh function, or a ReLU (rectified linear unit) function (ƒ(x)=max(0, x)). In addition, the “leaky” ReLU function (ƒ(x)=max(0.01*x, x)) has also been proposed, which replaces the flat section (i.e., x<0) of the ReLU function with a section that has a slight slope, usually 0.01, though the actual slope is trainable in some embodiments. In some embodiments, the activation functions can be other types of functions, including gaussian functions and periodic functions.

Before a multi-layer network can be used to solve a particular problem, the network is put through a supervised training process that adjusts the network's configurable parameters (e.g., the weight coefficients, and additionally in some cases the bias factor). The training process iteratively selects different input value sets with known output value sets. For each selected input value set, the training process typically (1) forward propagates the input value set through the network's nodes to produce a computed output value set and then (2) back-propagates a gradient (rate of change) of a loss function (output error) that quantifies the difference between the input set's known output value set and the input set's computed output value set, in order to adjust the network's configurable parameters (e.g., the weight values).

In some embodiments, training the neural network involves defining a loss function (also called a cost function) for the network that measures the error (i.e., loss) of the actual output of the network for a particular input compared to a pre-defined expected (or ground truth) output for that particular input. During one training iteration (also referred to as a training epoch), a training dataset is first forward-propagated through the network nodes to compute the actual network output for each input in the data set. Then, the loss function is back-propagated through the network to adjust the weight values in order to minimize the error (e.g., using first-order partial derivatives of the loss function with respect to the weights and biases, referred to as the gradients of the loss function). The accuracy of these trained values is then tested using a validation dataset (which is distinct from the training dataset) that is forward propagated through the modified network, to see how well the training performed. If the trained network does not perform well (e.g., have error less than a predetermined threshold), then the network is trained again using the training dataset. This cyclical optimization method for minimizing the output loss function, iteratively repeated over multiple epochs, is referred to as stochastic gradient descent (SGD).

In some embodiments the neural network is a deep aggregation network, which is a stateless network that uses spatial residual connections to propagate information across different spatial feature scales. Information from different feature scales can branch-off and re-merge into the network in sophisticated patterns, so that computational capacity is better balanced across different feature scales. Also, the network can learn an aggregation function to merge (or bypass) the information instead of using a non-learnable (or sometimes a shallow learnable) operation found in current networks.

Deep aggregation networks include aggregation nodes, which in some embodiments are groups of trainable layers that combine information from different feature maps and pass it forward through the network, skipping over backbone nodes. Aggregation node designs include, but are not limited to, channel-wise concatenation followed by convolution (e.g., DispNet), and element-wise addition followed by convolution (e.g., ResNet).

The term “computer” is intended to have a broad meaning that may be used in computing devices such as, e.g., but not limited to, standalone or client or server devices. The computer may be, e.g., (but not limited to) a personal computer (PC) system running an operating system such as, e.g., (but not limited to) MICROSOFT® WINDOWS®; NT/98/2000/XP/Vista/Windows 7/8/etc. available from MICROSOFT® Corporation of Redmond, Wash., U.S.A. or an Apple computer executing MACR OS from Apple® of Cupertino, Calif., U.S.A. However, the invention is not limited to these platforms. Instead, the invention may be implemented on any appropriate computer system running any appropriate operating system. In one illustrative embodiment, the present invention may be implemented on a computer system operating as discussed herein. The computer system may include, e.g., but is not limited to, a main memory, random access memory (RAM), and a secondary memory, etc. Main memory, random access memory (RAM), and a secondary memory, etc., may be a computer-readable medium that may be configured to store instructions configured to implement one or more embodiments and may comprise a random-access memory (RAM) that may include RAM devices, such as Dynamic RAM (DRAM) devices, flash memory devices, Static RAM (SRAM) devices, etc.

The secondary memory may include, for example, (but is not limited to) a hard disk drive and/or a removable storage drive, representing a floppy diskette drive, a magnetic tape drive, an optical disk drive, a compact disk drive CD-ROM, flash memory, etc. The removable storage drive may, e.g., but is not limited to, read from and/or write to a removable storage unit in a well-known manner. The removable storage unit, also called a program storage device or a computer program product, may represent, e.g., but is not limited to, a floppy disk, magnetic tape, optical disk, compact disk, etc. which may be read from and written to the removable storage drive. As will be appreciated, the removable storage unit may include a computer usable storage medium having stored therein computer software and/or data.

In alternative illustrative embodiments, the secondary memory may include other similar devices for allowing computer programs or other instructions to be loaded into the computer system. Such devices may include, for example, a removable storage unit and an interface. Examples of such may include a program cartridge and cartridge interface (such as, e.g., but not limited to, those found in video game devices), a removable memory chip (such as, e.g., but not limited to, an erasable programmable read only memory (EPROM), or programmable read only memory (PROM) and associated socket, and other removable storage units and interfaces, which may allow software and data to be transferred from the removable storage unit to the computer system.

The computer may also include an input device may include any mechanism or combination of mechanisms that may permit information to be input into the computer system from, e.g., a user. The input device may include logic configured to receive information for the computer system from, e.g. a user. Examples of the input device may include, e.g., but not limited to, a mouse, pen-based pointing device, or other pointing device such as a digitizer, a touch sensitive display device, and/or a keyboard or other data entry device (none of which are labeled). Other input devices may include, e.g., but not limited to, a biometric input device, a video source, an audio source, a microphone, a web cam, a video camera, and/or other camera. The input device may communicate with a processor either wired or wirelessly.

The computer may also include output devices which may include any mechanism or combination of mechanisms that may output information from a computer system. An output device may include logic configured to output information from the computer system. Embodiments of output device may include, e.g., but not limited to, display, and display interface, including displays, printers, speakers, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), etc. The computer may include input/output (I/O) devices such as, e.g., (but not limited to) communications interface, cable and communications path, etc. These devices may include, e.g., but are not limited to, a network interface card, and/or modems. The output device may communicate with processor either wired or wirelessly. A communications interface may allow software and data to be transferred between the computer system and external devices.

The term “data processor” is intended to have a broad meaning that includes one or more processors, such as, e.g., but not limited to, local processors or processors that are connected to a communication infrastructure (e.g., but not limited to, a communications bus, cross-over bar, interconnect, or network, etc.). The term data processor may include any type of processor, microprocessor and/or processing logic that may interpret and execute instructions (e.g., for example, a field programmable gate array (FPGA)). The data processor may comprise a single device (e.g., for example, a single core) and/or a group of devices (e.g., multi-core). The data processor may include logic configured to execute computer-executable instructions configured to implement one or more embodiments. The instructions may reside in main memory or secondary memory. The data processor may also include multiple independent cores, such as a dual-core processor or a multi-core processor. The data processors may also include one or more graphics processing units (GPU) which may be in the form of a dedicated graphics card, an integrated graphics solution, and/or a hybrid graphics solution. The data processor may be onboard, external to other components, or both. Various illustrative software embodiments may be described in terms of this illustrative computer system. After reading this description, it will become apparent to a person skilled in the relevant art(s) how to implement the invention using other computer systems and/or architectures.

The term “data storage device” is intended to have a broad meaning that includes removable storage drive, a hard disk installed in hard disk drive, flash memories, removable discs, non-removable discs, etc. In addition, it should be noted that various electromagnetic radiation, such as wireless communication, electrical communication carried over an electrically conductive wire (e.g., but not limited to twisted pair, CAT5, etc.) or an optical medium (e.g., but not limited to, optical fiber) and the like may be encoded to carry computer-executable instructions and/or computer data that embodiments of the invention on e.g., a communication network. These computer program products may provide software to the computer system. It should be noted that a computer-readable medium that comprises computer-executable instructions for execution in a processor may be configured to store various embodiments of the present invention.

### REFERENCES

1. Gilbert K, Forsch N, Hegde S, Mauger C, Omens J H, Perry J C, Pontré B, Suinesiaputra A, Young A A, McCulloch A D. Atlas-Based Computational Analysis of Heart Shape and Function in Congenital Heart Disease. J Cardiovasc Transl 2018:11:123-132.
2. Medrano-Gracia P, Cowan B R, Bluemke D A, Finn J P, Kadish A H, Lee D C, Lima J A, Suinesiaputra A, Young A A. Atlas-based analysis of cardiac shape and function: correction of regional shape bias due to imaging protocol for population studies. J Cardiov Magn Reson 2013:15:80.
3. Bai W, Shi W, Marvao A de, Dawes T J W, O'Regan D P, Cook S A, Rueckert D. A bi-ventricular cardiac atlas built from 1000+high resolution MR images of healthy subjects and an analysis of shape and motion. Med Image Anal 2015:26:133-145.
4. Buckner R L, Head D, Parker J, Fotenos A F, Marcus D, Morris J C, Snyder A Z. A unified approach for morphometric and functional data analysis in young, old, and demented adults using automated atlas-based head size normalization: reliability and validation against manual measurement of total intracranial volume. Neuroimage 2004:23:724-738.
5. Duchateau N, Craene M D, Piella G, Silva E, Doltra A, Sitges M, Bijnens B H, Frangi A F. A spatiotemporal statistical atlas of motion for the quantification of abnormal myocardial tissue velocities. Med Image Anal 2011:15:316-328.
6. Redheuil A, Wu C O, Kachenoura N, Ohyama Y, Yan R T, Bertoni A G, Hundley G W, Duprez. D A, Jacobs D R, Daniels L B, Darwin C, Sibley C, Bluemke D A, Lima J A C. Proximal Aortic Distensibility Is an Independent Predictor of All-Cause Mortality and Incident CV Events The MESA Study. J Am Coll Cardiol 2014:64:2619-2629.
7. Ohyama Y, Ambale-Venkatesh B, Noda C, Kim J-Y, Tanami Y, Teixido-Tura G, Chugh A R, Redheuil A, Liu C-Y, Wu C O, Hundley W G, Bluemke D A, Guallar E, Lima J A C. Aortic Arch Pulse Wave Velocity Assessed by Magnetic Resonance Imaging as a Predictor of Incident Cardiovascular Events. Hypertension 2017:70:524-530.
8. Sutton-Tyrrell K, Najjar S S, Boudreau R M, Venkitachalam L, Kupelian V, Simonsick E M, Havlik R, Lakatta E G, Spurgeon H, Kritchevsky S, Pahor M, Bauer D, Newman A, Study H A. Elevated Aortic Pulse Wave Velocity, a Marker of Arterial Stiffness, Predicts Cardiovascular Events in Well-Functioning Older Adults. Circulation 2005:111:3384-3390.
9. Ohyama Y, Ambale-Venkatesh B, Noda C, Chugh A R, Teixido-Tura G, Kim J-Y, Donekal S, Yoneyama K, Gjesdal O, Redheuil A, Liu C-Y, Nakamura T, Wu C O, Hundley W G, Bluemke D A, Lima J A C. Association of Aortic Stiffness With Left Ventricular Remodeling and Reduced Left Ventricular Function Measured by Magnetic Resonance Imaging. Circulation Cardiovasc Imaging 2018:9.
10. Redheuil A, Yu W-C, Mousseaux E, Harouni A A, Kachenoura N, Wu C O, Bluemke D, Lima J A C. Age-Related Changes in Aortic Arch Geometry Relationship With Proximal Aortic Function and Left Ventricular Mass and Remodeling. J Am Coll Cardiol 2011:58:1262-1270.
11. Fernandes V R S, Polak J F, Cheng S, Rosen B D, Carvalho B, Nasir K, McClelland R, Hundley G, Pearson G, O'Leary D H, Bluemke D A, Lima J A C. Arterial Stiffness Is Associated With Regional Ventricular Systolic and Diastolic Dysfunction. Arteriosclerosis Thrombosis Vasc Biology 2008:28:194-201.
12. Mitchell G F, Hwang S-J, Vasan R S, Larson M G, Pencina M J, Hamburg N M, Vita J A, Levy D, Benjamin E J. Arterial Stiffness and Cardiovascular Events. Circulation 2010:121:505-511.
13. Mitchell G F, Tardif J-C, Arnold J M O, Marchiori G, O'Brien T X, Dunlap M E, Pfeffer M A. Pulsatile Hemodynamics in Congestive Heart Failure. Hypertension 2001:38:1433-1439.
14. Desai A S, Mitchell G F, Fang J C, Creager M A. Central Aortic Stiffness is Increased in Patients With Heart Failure and Preserved Ejection Fraction. J Card Fail 2009:15:658-664.
15. Hundley W G, Kitzman D W, Morgan T M, Hamilton C A, Darty S N, Stewart K P, Herrington D M, Link K M, Little W C. Cardiac cycle-dependent changes in aortic area and distensibility are reduced in older patients with isolated diastolic heart failure and correlate with exercise intolerance. J Am Coll Cardiol 2001:38:796-802.
16. Bruse J L, McLeod K, Biglino G, Ntsinjana H N, Capelli C, Hsia T-Y, Sermesant M, Pennec X, Taylor A M, Schievano S, Group M of CHA (MOCHA) C. A statistical shape modelling framework to extract 3D shape biomarkers from medical imaging data: assessing arch morphology of repaired coarctation of the aorta. Bmc Med Imaging 2016:16:40.
17. Bruse J L, Zuluaga M A, Khushnood A, McLeod K, Ntsinjana H N, Hsia T-Y, Sermesant M, Pennec X, Taylor A M, Schievano S. Detecting Clinically Meaningful Shape Clusters in Medical Image Data: Metrics Analysis for Hierarchical Clustering Applied to Healthy and Pathological Aortic Arches. Ieee T Bio-med Eng 2017:64:2373-2383.
18. Schnell S, Smith D A, Barker A J, Entezari P, Honarmand A R, Carr M L, Malaisrie S C, McCarthy P M, Collins J, Carr J C, Markl M. Altered aortic shape in bicuspid aortic valve relatives influences blood flow patterns. European Hear J Cardiovasc Imaging 2016:17:1239-1247.
19. Bensalah M Z, Bollache E, Kachenoura N, Giron A, Cesare A D, Macron L, Lefort M, Redheuil A, Redheuill A, Mousseaux E. Geometry is a major determinant of flow reversal in proximal aorta. Am J Physiol-heart C 2014:306:H1408-H1416.
20. Bensalah Z M, Bollache E, Kachenoura N, Cesare A D, Redheuil A, Mousseaux E. Ascending aorta backward flow parameters estimated from phase-contrast cardiovascular magnetic resonance data: new indices of arterial aging. J Cardiov Magn Reson 2012:14:P128.
21. Westerhof B E, Westerhof N. Magnitude and return time of the reflected wave. J Hypertens 2012:30:932-939.
22. Jarvis K, Soulat G, Scott M, Vali A, Pathrose A, Syed A A, Kinno M, Prabhakaran S, Collins J D, Markl M. Investigation of Aortic Wall Thickness, Stiffness and Flow Reversal in Patients With Cryptogenic Stroke: A 4D Flow MRI Study. J Magn Reson Imaging 2020:
23. Bouaou K, Bargiotas I, Dietenbeck T, Bollache E, Soulat G, Craiem D, Houriez-Gombaud-Saintonge S, Cesare A D, Gencer U, Giron A, Redheuil A, Messas E, Lucor D, Mousseaux E, Kachenoura N. Analysis of aortic pressure fields from 4D flow MRI in healthy volunteers: Associations with age and left ventricular remodeling. J Magn Reson Imaging 2019:50:982-993.
24. Houriez-Gombaud-Saintonge S, Mousseaux E, Bargiotas I, Cesare A D, Dietenbeck T, Bouaou K, Redheuil A, Soulat G, Giron A, Gencer U, Craiem D, Messas E, Bollache E, Chenoune Y, Kachenoura N. Comparison of different methods for the estimation of aortic pulse wave velocity from 4D flow cardiovascular magnetic resonance. J Cardiov Magn Reson 2019:21:75.
25. Ferdian E, Suinesiaputra A, Dubowitz D J, Zhao D, Wang A, Cowan B, Young A A. 4DFlow Net: Super-Resolution 4D Flow MRI Using Deep Learning and Computational Fluid Dynamics. Aip Conf Proc 2020:8:138.
26. Vishnevskiy V, Walheim J, Kozerke S. Deep variational network for rapid 4D flow MRI reconstruction. Nat Mach Intell 2020:2:228-235.
27. Berhane H, Scott M, Elbaz M, Jarvis K, Mccarthy P, Carr J, Malaisrie C, Avery R, Barker A J, Robinson J D, Rigsby C K, Markl M. Fully automated 3D aortic segmentation of 4D flow MRI for hemodynamic analysis using deep learning. Magnet Reson Med 2020:84:2204-2218.
28. Tesche C, Cecco C N D, Baumann S, Renker M, McLaurin T W, Duguay T M, Bayer R R, Steinberg D H, Grant K L, Canstein C, Schwemmer C, Schoebinger M, Itu L M, Rapaka S, Sharma P, Schoepf U J. Coronary CT Angiography-derived Fractional Flow Reserve: Machine Learning Algorithm versus Computational Fluid Dynamics Modeling. Radiology 2018:288:64-72.
29. Gharib M, Beizaie M. Correlation Between Negative Near-Wall Shear Stress in Human Aorta and Various Stages of Congestive Heart Failure. Ann Biomed Eng 2003:31:678-685.
30. Knobelsdorff-Brenkenhoff F von, Karunaharamoorthy A, Trauzeddel R F, Barker A J, Blaszczyk E, Markl M, Schulz-Menger J. Evaluation of Aortic Blood Flow and Wall Shear Stress in Aortic Stenosis and Its Association With Left Ventricular Remodeling. Circulation Cardiovasc Imaging 2018:9:e004038.
31. Qi C R, Su H, Mo K, Guibas L J. PointNet: Deep Learning on Point Sets for 3D Classification and Segmentation. Arxiv 2016:
32. Qi C R, Yi L, Su H, Guibas L J. PointNet++: Deep Hierarchical Feature Learning on Point Sets in a Metric Space. Arxiv 2017:
33. Pascaner A F, Houriez-Gombaud-Saintonge S, Craiem D, Gencer U, Casciaro M E, Charpentier E, Bouaou K, Cesare A D, Dietenbeck T, Chenoune Y, Kachenoura N, Mousseaux E, Soulat G, Bollache E. Comprehensive assessment of local and regional aortic stiffness in patients with tricuspid or bicuspid aortic valve aortopathy using magnetic resonance imaging. Int J Cardiol 2021:326:206-212.
Nair, Vinod and Hinton, Geoffrey E., “Rectified linear units improve restricted Boltzmann machines,” ICML, pp. 807-814, 2010.
He, Kaiming, Zhang, Xiangyu, Ren, Shaoqing, and Sun, Jian, “Delving deep into rectifiers: Surpassing human-level performance on imagenet classification,” arXiv preprint arXiv: 1502.01852, 2015.
Mayer, Nikolaus, Ilg, Eddy, Häusser, Philip, Fischer, Philipp, Cremers, Daniel, Dosovitskiy, Alexey, and Brox, Thomas, “A Large Dataset to Train Convolutional Networks for Disparity, Optical Flow, and Scene Flow Estimation,” arXiv preprint arXiv: 1512.02134, 2015.
He, Kaiming, Zhang, Xiangyu, Ren, Shaoqing, and Sun, Jian, “Deep Residual Learning for Image Recognition,” arXiv preprint arXiv: 1512.03385, 2015.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described. Moreover, features described in connection with one embodiment may be used in conjunction with other embodiments, even if not explicitly stated above.

1. A method for characterizing a blood vessel, comprising:
receiving anatomical information of the blood vessel;
receiving four-dimensional flow data of a plurality of blood vessels from a plurality of subjects;
using the four-dimensional flow data, training a flow model to determine blood vessel properties; and
using the anatomical information of the blood vessel as an input to the trained flow model, estimating one or more properties of the blood vessel.

2. The method of claim 1, wherein the anatomical information comprises shape information of the blood vessel.

3. The method of claim 1, wherein the anatomical information of the blood vessel is generated from a segmentation process applied to one or more images of the blood vessel that are acquired using one of a doppler ultrasound scanner, a computed tomography scanner, and a magnetic resonance scanner.

4. The method of claim 1, wherein the one or more properties of the blood vessel comprise at least one of a wall shear stress of the blood vessel expressed in units of force per unit area, a wall stiffness of the blood vessel expressed in units of pulse wave velocity, a vorticity of the blood vessel expressed in units of inverse time, a velocity of blood within the blood vessel expressed in units of distance over time, and a flow of blood within the blood vessel expressed in units of volume over time.

5. The method of claim 1, wherein the blood vessel is one of an aorta, a coronary artery, a carotid artery, a superior vena cava, and an inferior vena cava.

6. The method of claim 1, further comprising receiving one or more two-dimensional flow measurements of blood that is one of entering and exiting the blood vessel,
wherein the one or more two-dimensional flow measurements are a further input to the trained flow model.

7. The method of claim 1, wherein the four-dimensional flow data of the plurality of blood vessels comprises data acquired from the plurality of subjects using magnetic resonance imaging.

8. The method of claim 1, wherein the four-dimensional flow data of the plurality of blood vessels comprises simulated data based on the plurality of subjects.

9. A system for characterizing a blood vessel, comprising:
an imaging system; and
a data processor configured to communicate with said imaging system to receive anatomical information of the blood vessel, said data processor being further configured to:
receive four-dimensional flow data of a plurality of blood vessels from a plurality of subjects;
using the four-dimensional flow data, train a flow model to determine blood vessel properties; and
using the anatomical information of the blood vessel as an input to the trained flow model, estimate one or more properties of the blood vessel.

10. The system of claim 9, wherein the anatomical information comprises shape information of the blood vessel.

11. The system of claim 9, wherein the anatomical information of the blood vessel is generated from a segmentation process applied to one or more images of the blood vessel that are acquired using the imaging system.

12. The system of claim 9, wherein the imaging system comprises an image processor and at least one of a doppler ultrasound scanner, a computed tomography scanner, and a magnetic resonance scanner.

13. The system of claim 9, wherein the one or more properties of the blood vessel comprise at least one of a wall shear stress of the blood vessel expressed in units of force per unit area, a wall stiffness of the blood vessel expressed in units of pulse wave velocity, a vorticity of the blood vessel expressed in units of inverse time, a velocity of blood within the blood vessel expressed in units of distance over time, and a flow of blood within the blood vessel expressed in units of volume over time.

14. The system of claim 9, wherein the blood vessel is one of an aorta, a coronary artery, a carotid artery, a superior vena cava, and an inferior vena cava.

15. The system of claim 9, wherein the data processor is further configured to receive from the imaging system one or more two-dimensional flow measurements of blood that is one of entering and exiting the blood vessel,
wherein the one or more two-dimensional flow measurements are a further input to the trained flow model.

16. The system of claim 9, wherein the four-dimensional flow data of the plurality of blood vessels comprises data acquired from the plurality of subjects using magnetic resonance imaging.

17. The system of claim 9, wherein the four-dimensional flow data of the plurality of blood vessels comprises simulated data based on the plurality of subjects.

18. A non-transitory machine-readable medium storing a program for characterizing a blood vessel, which when executed by a data processor, configures said data processor to:
receive anatomical information of the blood vessel;
receive four-dimensional flow data of a plurality of blood vessels from a plurality of subjects;
using the four-dimensional flow data, train a flow model to determine blood vessel properties; and
use the anatomical information of the blood vessel as an input to the trained flow model, estimating one or more properties of the blood vessel.

19. The machine-readable medium of claim 18, wherein the anatomical information comprises shape information of the blood vessel.

20. The machine-readable medium of claim 18, wherein the anatomical information of the blood vessel is generated from a segmentation process applied to one or more images of the blood vessel that are acquired using one of a doppler ultrasound scanner, a computed tomography scanner, and a magnetic resonance scanner.

21.-25. (canceled)
